(19) United States
(12) Patent Application Publication
(10) Pub. No.: US 2024/0050503 A1
(43) Pub. Date: Feb. 15, 2024

(54) FLOWER ESSENCE-CONTAINING MEDICAMENTS

(71) Applicants: Clare G. Harvey, Sherbourne (GB); Dori Naerbo, Sirevag (NO)

(72) Inventors: Clare G. Harvey, Sherbourne (GB); Dori Naerbo, Sirevag (NO)

(21) Appl. No.: 17/817,028

(22) Filed: Aug. 3, 2022

(51) Int. Cl. A61K 36/14; A61K 36/73; A61K 9/06; A61K 35/06; A61K 36/738; A61K 36/752

(52) U.S. Cl. CPC A61K 36/14; A61K 36/73; A61K 9/06; A61K 35/06; A61K 36/738; A61K 36/752

(57) ABSTRACT

A method can relate to the use of flower essence-containing medicaments, particularly those that are useful for the alleviation of stress. More particularly, though not exclusively, a method can relate to topical applications including creams, such as facial and body cream, oils, spritzers and toners and serums, for oral and/or external application, that are useful for the alleviation of stress. Compositions provide a combination of advantageous properties of a cosmetic composition including one or more of Appearance, Odour, Ease of Application, Ease of Absorbency, Absorbency time, Greasiness (lower score if greasier), Irritation, Skin, Feel (softness) and Skin Moisturisation. Compositions can include one or more plant stem cell extracts in combination with one or more flower essences. A composition can preferably include extracts from Fitzroya cupressoide.

### RELATED APPLICATION

This application is related to a GB application having patent application number GB2118418.9, entitled FLOWER ESSENCE-CONTAINING MEDICAMENTS, to Clare G. Harvey and Dr. Dori Naerbo, which is incorporated by reference herein.

### BACKGROUND

“Stress” is defined to be any interference that disturbs a person's healthy mental and physical well-being. Our stress response is relative and highly individual. Stress is being recognized as a medical condition. Scientists at Harvard University researching into the effect of stress on the body have discovered shared interactive chemicals and links between the mind and the body, especially in relation to the nervous system, immune and endocrine system as well as the skin. In fact, stress has been dubbed the “Health Epidemic of the 21st Century” by the World Health Organization. According to the Centers for Disease Control and Prevention, stress-related disorders and diseases such as heart disease, obesity and diabetes have been on the rise in the whole population for decades. A similar pattern is emerging in stress-related mental health. Studies and research show, that the vast majority of people are feeling stressed, anxious and overwhelmed. Chronic stress has become the new-norm reaching epidemic proportions. And it shows up in our bodies, even before we get sick. Even though we're more connected than ever before, loneliness is now considered a major stress factor.

Stress raises your cortisol levels. Researchers have known for years that elevated cortisol levels sustained over time causes damage. Examples of the damaging effects of chronic stress in the brain include: free radicals harming brain cells; making a person become more forgetful and emotional; creating a vicious cycle of fear and anxiety; halting the production of new brain cells; depleting critical brain chemicals causing depression; increasing risk for mental illnesses of all kinds; shrinking your brain; allowing toxins into your brain; increasing your risk of dementia and Alzheimer's; promoting brain cells to commit suicide; and chronic stress contributing to brain inflammation and depression. Examples of physical effects as a result of chronic stress in the brain include: lowering your immune system; weight gain; increased blood pressure; lowering bone density; high cholesterol; increased risk of heart disease; erectile dysfunction; increased risk of heart attack; low sex drive; headaches; insomnia; and digestive problems.

A person may experience stress in response to a wide range of physical and emotional stimuli, including physical violence, internal conflicts and significant life events such as death of a loved one, birth of a baby, or divorce. Some people are more susceptible than others to stress-related medical problems. When faced with a stressful situation the body responds by increasing production of certain hormones such as cortisol and adrenaline. These hormones lead to changes in heart rate, blood pressure, metabolism, and physical activity designed to improve overall performance. However, at a certain level, they disrupt an individual's ability to cope. Continued exposure to stress often leads to mental and physical symptoms, such as anxiety and depression, indigestion, palpitations, low libido, insomnia, weight gain or loss, and muscular aches and pains.

It is now accepted and recognized that there is a direct correlation between the mind, and the body. Whether positive or negative, stress has a direct beneficial or detrimental effect on the physiology of the body.

Various treatments are used to assist people suffering from stress, including pharmaceutical drug therapy, complementary medicine, relaxation techniques such as yoga and meditation, aromatherapy and other therapeutic modalities.

Aromatherapy involves a range of treatments using essential oils and flower essences that are extracted from plants. Essential oils are known to contain the plant actives that have a beneficial effect of repairing the cellular structure of the skin and helping to stimulate the growth of new cells; flower essences, on the other hand, are known as the flowers liquid energy and have the extraordinary ability directly to enhance and alter our emotional response to ourselves and the world, bringing relief from unsettling moods and emotions such as fear, anxiety, guilt, anger and melancholy, and helping with the development of a positive and stress-free attitude towards our life. It is well known that a happy self-image has a powerful physiological effect that adds to the inner glow of health vitality. Many ancient civilizations, particularly the Chinese, documented the use of such materials in treating different disorders, and some aromatherapy treatments are known to have effect on stress-related conditions. Indeed, the Australian Aborigines practiced one of the most ancient forms of flower essence therapy. Around 10,000 years ago they were gathering dew-drenched flowers and either drinking the dew or placing the flowers in their mouths. If the flowers were inedible, then they would simply sit among them. They found that the flowers could restore a person's mental attitude. If someone were afraid, they would gain courage, if angry they would find peace, if sick would become well again. It is believed that Native Americans, Minoans and ancient Egyptians harnessed the healing powers of flowers in similar ways.

Whilst the aforementioned flower essences are well established, such as disclosed in GB2409645B by the same inventor, the field of alternative (complimentary) therapies continues to advance and stem cell properties have come to the fore is also being beneficial.

Stem cells are used in regenerative medicine and as a source of undifferentiated cellular material provide the opportunity for rebuilding tissues as the stem cells as the stem cells differentiate and can be incorporated into the tissue thus regenerating it. “Anti-Aging Properties of Plant Stem Cell Extracts” to Miastkowska, M. et al in Cosmetics (2018), Vol. 5, No. 55 discloses skin aging is a complex process which involves all the layers of the epidermis and dermis. Plant stem cells show outstanding anti-aging properties, as they can, among other activities, stimulate fibroblasts to synthesise collagen, which, in turn, stimulates skin regeneration. One of the most important agents which give anti-aging properties to plant stem cell extracts is kinetin (6-furfuryladenine). It enables cells to remove the excess of free radicals to protect them from oxidative stress. “Plant stem cells in cosmetics: current trends and future directions” to Trehan, S. et al in Future Sci OA. (2017) Vol. 3, No. 4, discloses that plant regeneration at the cellular and tissue level is a unique process, and that the stem cells in plants have properties that help stimulate and regenerate plants after injury. “Wound Healing Effects of Rose Placenta in a Mouse Model of Full-Thickness Wounds” to Kim, Y. W. et al in Arch Plast Surg. (2015), Vol. 42, No. 6, pp. 686-694, discloses Rosa damascena, a type of herb, which has been used for wound healing in Eastern folk medicine, and which was used to evaluate the effectiveness of rose placenta in a full-thickness wound model in mice. “Stem bark and flower extracts of Vismia cauliflora: modulation of oxidative burst in human neutrophils' and inhibition of oxidative damage in human erythrocytes” to Ribeiro, A. B. et al in Free Radical Biology and Medicine (2014), Vol. 75, Supp. 1, pp. S45-S46, discloses Vismia cauliflora, an Amazonian plant traditionally used to treat dermatosis and inflammatory processes of the skin by indigenous populations.

As will be appreciated, whatever the specific mechanism of action whether in alternative therapies or classical medicine there are perceived benefits in providing compositions containing components which provide beneficial effects, whether through a physiological mechanism or a psychological mechanism. There is therefore a need to provide compositions suitable for delivery to the human body comprising the aforementioned ingredients. The provision of such compositions which are physiologically acceptable, have physical properties to enable them to be readily delivered to a point of use, and are physically, chemically and biologically stable is therefore advantageous. Each one of these individual properties is in itself advantageous.

A specific form of plant of interest in providing plant-based remedies are orchids. There are many hundreds of varieties of orchids indigenous to many countries or regions of the world. Some orchid essences, such as Caladenia latifolia, Chytroglossa aurata and Hofmeister ellaru microscopica, are known to reduce stress levels when administered orally or topically.

### SUMMARY

Technology and techniques relate to a topical-application composition suitable for reducing or alleviating stress, a composition can include one or more plant stem cell extracts in combination with one or more flower essences.

Preferably the one or more plant stem cell extracts are selected from all monocot and dicot flowering plants, shrubs and trees.

Preferably the one or more flower essences are selected from essences, such as Acineta superba, Amazonas, Angulo aclowesii, Caladenia latifolia, Cattleya warscewiczii, Cauleyatrianae, Chytroglossa aurata, Cymbidium lowianum, Epidendrum chioneum, Epidendrum prismatocarpum, Epidendrum secundum, Fitzroya cupressoide, Hofmeister ellaru microscopica, Laeliocattleya anceps clara, Laeliocattleya hybr., Miltonia phalaenopsis, Oncidium abortivum, Oncidium altissimum, Oncidium incurvum, Oncidium lanceanum, Oncidium sphacelatum, Orchis mascula, Paphiopedilum harrysianum, Paphiopedilum insigne, Stanhopeawardii, Vanda tricolor, Victoria amazonica and combinations thereof.

One particular flower essence species of interest is Fitzroya cupressoide belonging to the Cupresaceae family. It is a monotypic genus, known as Alerce or Patagonian Alerce native to Argentina and Chile. It is a slow growing species, and it has been reported that a specimen over 3600 years has been found in the Alerce Coastal National Park in Chile.

Few studies on the physiology or metabolism of Fitzroya cupressoides have been made. “Sesquiterpene alcohols from foliage of Fitzroya cupressoides” to Cool, L. G in Phytochemistry (1996), Volume 42, No. 4, pp. 1015-1019 discloses a new trans-fused himachal-4-en-1-ol which was isolated from the foliage of the α-longipinene-producing chemotype of Fitzroya cupressoides, and characterized by 1D- and 2D-NMR by partial synthesis. U.S. Pat. No. 5,955,621 discloses Alerce seed and sprout cells which were used to evaluate the presence of taxane compounds (for antineoplastic uses) in plant cells.

An aqueous extract obtained from Alerce cells, having pharmaceutical and cosmetic properties, particularly in skin regeneration and anti-aging effect is known (WO2020/075074A1). The extract is useful in developing products for the treatment and protection of the skin in order to promote health, and reduce the appearance of wrinkles and expression lines, as well as tissue regeneration in cases of recent injury or scarring.

It has now been surprisingly discovered that extracts of Fitzroya cupressoides essences can be used in the preparation of medicaments for reducing stress levels.

Most preferably, the one or more flower essences are selected from Amazonas, Angulo aclowesii, Caladenia latifolia, Cattleya warscewiczii, Chytroglossa aurata, Epidendrum chioneum, Epidendrum secundum, Fitzroya cupressoide, Hofmeister ellaru microscopica, Miltonia phalaenopsis, Oncidium abortivum, Oncidium sphacelatum and combinations thereof.

Preferably the composition is an aqueous-based extract.

Preferably the composition is a cream.

Preferably the composition includes one or more optional berry extracts including, but not limited to, extracts of Maiqui berries.

The addition and inclusion of all monocot and dicot flowering plants, shrubs and trees, and their stem cells essence is envisaged. During the creation process of stem cell essence, naturally occurring stem cells may be present. Their robust mechanism for regeneration of tissue under stress, per techniques and technologies herein, expands and enhances the therapeutic action and value of all the medicament applications previously mentioned.

Plant stem cells are the very essence of the food we consume, air we breathe and fuel we burn. Hence, plant stem cells may be an important source to maintain homeostasis and optimum health. According to Current Biology, (Greb & Lohmann 2016) “In some long-lived plants, such as trees, plant stem cells remain active over hundreds or even thousands of years, revealing the exquisite precision in the underlying control of proliferation, self-renewal and differentiation.”

Plant stem cells are located in three places: 1) apical shoots of monocot and dicot flower plants, shrubs and trees which are located at just below the flowering shoot(s), 2) cambium of monocot and dicot flower plants, shrubs and trees which are located in the stems of flowering shoots, and 3) root apical meristems of monocot and dicot flower plants, shrubs and trees which are located in the roots of the flowering shoots.

The unique properties of plant stem cells have been a recent area of interest and focus both in developing new cosmetics. Every organism has stem cells and plant stem cells come from the stem of the plant. Plant regeneration at the cellular and tissue level is a unique process and the stem cells in plants have properties that help stimulate and regenerate themselves after injury. Plants are equipped with a robust mechanism for regeneration of their tissues under stress.

One of the ways to extract the stem cells is the plant is allowed to dry in order to activate the stem cells as this is the point that the plant produces more stem cells to heal itself, another method is to bruise or cut the root and allow the natural process of osmosis to occur, using the sun method of preparation. The process of osmosis is the dispersal of solvent molecules across a selectively or semi-permeable membrane where the molecules move from a higher concentrated solution to more dilute solutions. Where in this case this form of extraction can be enhanced by the presence of warmth of direct sun light. Cell division in plants occurs in regions called meristems. Cells of the meristem can differentiate to produce all types of plant cells at any time during the life of the plant. The main meri-stems are close to the tip of the shoot, and the tip of the root.

In a growing shoot, new cells are being produced continuously near the tip. As the cells become older, they move further away from the tip and become differentiated. Plant stem cells show outstanding anti-aging properties, as they can, among other activities, stimulate fibroblasts to synthesise collagen, which, in turn, stimulates skin regeneration. Skin aging is a complex process which involves all the layers of the epidermis and dermis. In order to slow skin aging, methods are researched which would strengthen and protect skin stem cells. Science is in search of the right method to stimulate the proliferation of epidermal stem cells. One of the most important agents which give anti-aging properties to plant stem cell extracts is kinetin (6-furfuryladenine).

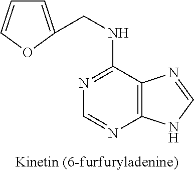 [SMILES: c1coc(CNc2ncnc3[nH]cnc23)c1]

This compound belongs to a cytokine group and is considered to be a strong antioxidant which protects protein and nucleic acids from oxidation and glycoxidation processes. It enables cells to remove the excess of free radicals to protect them from oxidative stress and all forms of skin stress in general.

Stem cell essences and stem cells of monocot and dicot flower plants, shrubs and trees are contained within the flower essence, which are an essential component. Part of the process of extracting the essences involve a simultaneous process of extracting the stem cells creating a symbiotic process.

Accordingly, various techniques and technologies described herein can provide the use of one or more essences in the preparation of medicaments for reducing stress levels. In another aspect, there is provided a medicament for use in topical applications including creams, such as facial and body cream, oils, spritzers and toners and serums, for oral and/or external application to the face, body, hair, eyebrows and eyelashes, that are useful for the alleviation of stress. All forms of cosmetics designed for men and woman are contemplated including, but not limited to, nail polish, eye shadows, lipsticks, foundation, blushes, powder essence, bronzer, highlighters and mascara, comprising one or more orchid essences, in addition to, precious metals, precious and semi-precious stones, crystals, gems in solution, powder, elixir and essence. Medicaments comprising one or more specific essences have strong mood enhancing properties and are thereby extremely effective at reducing stress levels in the human body. These highly beneficial properties of these specific orchid essences were hitherto unknown.

Applications of various techniques and/or technologies described herein can include one or more of the following features:

The medicaments can further include a flower remedy liquid essence, tincture, oral spray and body spritzers. These flower essence solutions can reduce stress and promote a sense of emotional and psychological well-being. When the medicaments measured by heart rate variability can demonstrate a reduction in biological age in comparison to chronological age.

The flower essence solutions can include a member selected from the group consisting of flower essences, adaptogens, polyphenols, antioxidants, herbal extract, vitamin, crystal essence, precious, semi-precious gems, and precious metal essences, mineral, herb, and combinations thereof.

The medicaments reduce stress, anxiety and enhance mood. As a result, it may improve sleep and accelerate skin repair, cellular regeneration, improve collagen, hair and nail growth.

The medicaments may optionally comprise one or more further non-orchid flower essences (hereafter termed “secondary flower essences”) one or more essential oils, one or more phytolaits, one or more whole perennial and annual plants, cereal grains, deciduous and evergreen in its entity including the seeds, roots, root apices, stems, leaves, meristems, apical shoots, and complete flowers, one or more of the proteaceae family, one or more of the coniferophyta family, one or more of the rosaceae family, one or more of the anthophyta family, one or more phytocermides, one or more peptides, one or more plant stem cells, one or more plant stem cell signalling peptides, one or more berries, one or more seed oils, one or more fruit cell extracts and fruit oils, one or more flower extracts, one or more leaf extract, one or plant stem cells extracts, one or more hydrolat flower waters, one or more tea extracts, one or more adaptogenic herbs, one or more of the algae family, one or more mushrooms, e.g. tremella, chaga, reishi, shiitake, and one or more monoils.

In each of the above aspects, the essences are selected from Acinetasuperba, Amazonas, Epidendrum secundum, Miltonia phalaenopsis, Oncidium incurvum, Stanhopeawardii, Oncidium lanceanum, Cymbidium lowianum, Epidendrum prismatocarpum, Cattleya warscewiczii, Vanda tricolor, Laeliocattleya hybr., Laeliocattleya anceps clara, Cauleyatrianae, Oncidium abortivum, Paphiopedilum harrysianum, Paphiopediluminsigne, Epidendrumchioneum, Angulo aclowesii, Orchismascula, Oncidium sphacelatum and Victoria amazonica. Preferred orchid essences are selected from Epidendrum secundum, Miltonia phalaenopsis, Epidendrum chioneum, Angulo aclowesii, Oncidium sphacelatum, Amazonas, Cattleya warscewiczii and Oncidium abortivum.

The secondary flower essences are selected from Aesculus hippocastanum, Bromus ramosus, Rosa canina, Salix vitellina, RQ5 (Cherry plum, clematis, impatiens, Rock rose and Star of Bethlehem) and RQ8 (Cherry plum, clematis, impatiens, Rock rose, Star of Bethlehem Lotus and Self-heal); essences from cacti and succulent plants such as: Caralluma russelliana, Cereus peruvianus, Ceropegia fusca, Cleistocactus ritterei, Cleistocactus stausii, Echinocactus grusonii, Echinocereus scheeri, Echinopsis oxygona, Ferocactus schwarzii, Hylocereus undatus, Mammillaria rubrograndis, Myitillocactus geometrizans, Opuntia cardiosperma, Opuntia dejecta, Orbea variegata, Pilosocereus pachycladus, Selenicereus grandiflorus, Seticereus icosagonus, Stenocereus marginatus and Stopelia desmetiana; rose essences such as Rosa roxbourghii, Rosa majalis, Rosa spinosissima, Rosa x pruhoniciana, Rosa rugosa “Sara van fleet”, Rosa rugosa “Souvenir de philemoncochet”, Rosa Moyesi; essences from Africa and the Canary islands such as Anagallis arvensis, Musa x paradisiacal “nana, Erica arborea, Plumbago auriculata, Eucalyptus globules, Monstera deliciosa, Sonchus acaulis, Geranium perforatum, K9, Canarina canariensis, Artemisia arvensis, Cocos nucifera, Mimosa pudica, Echium vulgare, Strelitzia reginae, Narcissus tazetta, Hibiscus rosa sinensis, Echium wildpretii, Euphorbia pulcherrima and Cistus albidus; and wild flower essences such as Aloe vera, Arnica montana, Ocimum basilicum, Borago officinalis, Prunella vulgaris, Ranunculus acris, Iris germanica, Hypericum perforatum, Verbascum thapsus, Lavandula officinalis, Taraxacu officinale, Viscum album, Rosmarinus officinalis, Trifolium pratense, Achillea millefolium, Helianthus annuus, Dicentra spectabilis, Viola hirta, Myosotis sylvatica, Zinnia elegans, Valeriano officinalis, Rubus fructicosus, Zantedeschia aethiopica, Anethum graveolens, Lilium bulbferum, Bellis perennis, Matricaria chamomilla, Polygonum bistorta, Cucurbita pepo, Nelumbo nucifera, Prunus amygdalus, Zea mays, Passiflora bryonioides, Ipomea purpurea, Achillea millefolium, Salvia officinalis, Epilobium angustifolium, Paradisea liliastrum, Allium angulosum, Daucus carota, Opuntia cardiospera, Seticereus icosagonus, Stapelia deshietiana, Rosa, Chinensis viridijlora, Ginkgo biloba, Citrus limon, Geranium perforatum, Cananna cananersis, Narassustuzetta, Hibiscus rosa-sinensis, Cistus allidus, Achillea millefolum, Salvia officinalis, Vinca minor, Echinaecea purpurea, Jepunbali, Jepunjawa, Aloe striata, Allium ursinum, Tussilago farara, Oenothera biennis, Capsicum annuum, Celendula officinalis, Mentha pulegium and Primula veris.

The preferred secondary flower essences are selected from Rosa canina, Echinocereus scheeri, Pilosocereus pachycladus, Rosa x pruhoniciana, Rosa Moyesii, Canarina, Canariensis, Strelitzia reginae, Aloe vera, Arnica montana, Prunella vulgaris, Zinnia elegans, Matricaria chamomilla, Nelumbo nucifera, Passiflora bryonioides, Paradisea liliastrum, Rosa spinosissima, Hibiscus Rosa, Jepunbali, Jepunjawa, Rosa roxbourghii, Orbea variegata, Cocos nucifera, Opuntia cardiosperma and Lilium bulbiferum.

Suitable essential oils include those from: Citrus reticulata, Chamaemelum, Geraniaceae such as Pelargonium gravea, Aurantium Bergamia, Lavandula angustiforlia, Rosa centifloria absolute, Jasminum officinale abs, Citrus paradisi, Cananga odorata, Buswellia sp., Citrus aurantifolia, riganum, Citrus aurantium, Cymbopogan martini, Ravensara aromatica, Santalum album, Santalum spicatum, Lavendula x intermedia, Cymbopogon citrates, Citrus reticulata, Melissa officinalis and Citrus sinesnsis.

Preferably, the amount of the one or more flower essences is present in an amount of at least at least 0.1% by volume, preferably at least 0.2% by volume, preferably at least 0.3% by volume, preferably at least 0.4% by volume, preferably at least 0.5% by volume, preferably at least 0.6% by volume, preferably at least 0.7% by volume, preferably at least 0.8% by volume, preferably at least 0.9% by volume or preferably at least 1.0% by volume. Preferably, the amount of the one or more flower essences is present in an amount of at most 9% by volume, preferably at most 9.5% by volume, preferably at most 10% by volume, preferably at most 10.5% by volume, preferably at most 11% by volume, preferably at most 11.5% by volume, preferably at most 12% by volume, preferably at most 12.5% by volume, preferably at most 13% by volume, preferably at most 13.5% by volume, preferably at most 14% by volume, preferably at most 14.5% by volume, preferably at most 15% by volume, preferably at most 15.5% by volume, preferably at most 16% by volume, preferably at most 16.5% by volume, preferably at most 17% by volume, preferably at most 17.5% by volume, preferably at most 18% by volume, preferably at most 18.5% by volume, preferably at most 19% by volume, preferably at most 19.5% by volume or preferably at most 20% by volume. More preferably, the amount of the one or more flower essences is present in the range 0.5-15% by volume. Most preferably, the amount of the one or more flower essences is present in the range 2-10% by volume.

Preferably, the amount of the one or more secondary flower essences is present in an amount of at least at least 0.1% by volume, preferably at least 0.2% by volume, preferably at least 0.3% by volume, preferably at least 0.4% by volume, preferably at least 0.5% by volume, preferably at least 0.6% by volume, preferably at least 0.7% by volume, preferably at least 0.8% by volume, preferably at least 0.9% by volume or preferably at least 1.0% by volume. Preferably, the amount of the one or more secondary flower essences is present in an amount of at most 9% by volume, preferably at most 9.5% by volume, preferably at most 10% by volume, preferably at most 10.5% by volume, preferably at most 11% by volume, preferably at most 11.5% by volume, preferably at most 12% by volume, preferably at most 12.5% by volume, preferably at most 13% by volume, preferably at most 13.5% by volume, preferably at most 14% by volume, preferably at most 14.5% by volume, preferably at most 15% by volume, preferably at most 15.5% by volume, preferably at most 16% by volume, preferably at most 16.5% by volume, preferably at most 17% by volume, preferably at most 17.5% by volume, preferably at most 18% by volume, preferably at most 18.5% by volume, preferably at most 19% by volume, preferably at most 19.5% by volume or preferably at most 20% by volume. More preferably, the amount of the one or more secondary flower essences is present in the range 0.5-15% by volume. Most preferably, the amount of the one or more secondary flower essences is present in the range 2-10% by volume.

Preferably, the amount of the one or more essential oils is present in an amount of at least at least 0.01% by volume, preferably at least 0.02% by volume, preferably at least 0.03% by volume, preferably at least 0.04% by volume, preferably at least 0.05% by volume, preferably at least 0.06% by volume, preferably at least 0.07% by volume, preferably at least 0.08% by volume, preferably at least 0.09% by volume or preferably at least 0.1% by volume. Preferably, the amount of the one or more essential oils is present in an amount of at most 1% by volume, preferably at most 1.5% by volume, preferably at most 2% by volume, preferably at most 2.5% by volume, preferably at most 3% by volume, preferably at most 3.5% by volume, preferably at most 4% by volume, preferably at most 4.5% by volume, preferably at most 5% by volume, preferably at most 5.5% by volume, preferably at most 6% by volume, preferably at most 6.5% by volume, preferably at most 7% by volume, preferably at most 7.5% by volume, preferably at most 8% by volume or preferably at most 8.5% by volume. More preferably, the amount of the one or more essential oils is present in the range 0.05-5% by volume. Most preferably, the amount of the one or more essential oils is present in the range 0.05-3% by volume. Highly preferably, the amount of the one or more essential oils is present in the range 0.1-1% by volume.

Phytolaits are plant milk that are rich in natural plant proteins, lipids and sugars. They are useful, particularly in cream formulations to be applied to the skin, because they have skin-hydrating, moistening and restructuring properties. Suitable phytolaits can be extracted from acacia flowers (Robinia pseudo-acacia), almonds, aloe vera, apricots, barley, blond pea, coconut, copalba, grape seeds, green tea, jasmine flowers, lotus flowers, lupine flowers, nectarines, oats, palms, passion flowers, soybeans and walnuts. Preferred phytolaits are extracted from acacia flowers and jasmine flowers.

The flower essences used may be extracted from flowers using any known technique such as, for example, the traditional Bach method in which the flowers are floated in a crystal bowl and left in sunlight for up to 2-3 hours. Mixing with an equal volume of alcohol or red shiso, which then preserves the resulting mother tincture.

Alternatively, a crystal method may be used in which the blooms are neither picked nor damaged. A mother tincture is obtained which is again preserved with an equal volume of alcohol or red shiso. The alcoholic or red shiso mother tincture is then generally used directly or in a diluted stock form. The medicaments described herein may be taken internally or applied externally to the body. Externally, they may be used pure or, for example, in the form of a soapy or aqueous emulsion, in an alcoholic solution, in an oil base, as a liniment, in overall or localized bathing, and in the form of creams, balms, lotions and spritzers. Internally, depending upon the case, dosage will vary between 5 and 20 drops of pure essence administered several times a day before or after meals, or between 20 and 30 drops of pure essence administered several times a day in water, under the tongue or directly on the wrist.

Preferably the medicament is in the form of a topical cream, such as a facial cream. Typically, the cream will comprise: one or more orchid flower essences, one or more secondary flower essences, one or more essential oils and one or more phytolaits.

The phytolaits are used to form a “base cream” and the flower essences, essential oils and any other ingredients are added to this. Fitzroya cupressoide (Alerce) may also be used as a base in a form of concentrated serum form for one or more formulations. The amount of the one or more orchid flower essence is preferably in the range 0.5-15% by volume of the base cream, 1-15% by volume is highly beneficial and 2-10% by volume is especially advantageous. These quantities are also typical for the amount of secondary flower essence used. The amounts of essential oil used are typical, such as from 0.05-5% by volume of the base cream, preferably 0.05-3% by volume and further preferably 0.1-1% by volume. 0.2% by volume is especially advantageous depending on the formulation requirements.

In an alternative embodiment, a medicament may also be formulated as a body oil in which the amount of phytolait is either reduced or replaced by one or more mono oils. A preferred mono oil is Tahitian coconut oil. Again, the flower essences and essential oils are added to the one or more mono oils in the same % by volume amounts described above.

Other ingredients may also be optionally included in the topical cream formulations, for example, manuka honey, herbal and floral waters, crystals and gems and precious and semi-precious metals such as gold and silver.

As explained, technology and techniques relate to the use of flower essence-containing medicaments, particularly those that are useful for the alleviation of stress. More particularly, though not exclusively, various examples relate to topical applications including creams, such as facial and body cream, oils, spritzers and toners and serums, for oral and/or external application, that are useful for the alleviation of stress.

Various technologies and techniques can be further understood with reference to the following Examples.

### EXAMPLES

Orchid flower essence-containing medicament Formulations 1-3 are detailed below. Formulations 1-3 are the basis for the spritzers and toners. These were made using conventional methods, which involve firstly forming a base cream from phytolait and then drop wise addition of the flower essences and essential oils in the amounts detailed below. Component parts are indicated in % amounts by volume, with the remainder of any solutions being made up with water.

Formulation 1

| | |
|---|---|
| Orchid essences | Fitzroya cupressoide, Epidendrum chioneum, |
| (5% by vol): | Miltonia phalaenopsis and Epidendrum Chioneum |
| Secondary flower | Amica montana, Prunellavulgaris, Matriciaria |
| essences | chamomilla, Zinnia, elegans, Rosa |
| (7% by vol.): | spinosissima, Hibiscus, Rosa and Jepunbali |
| Essential oils | Citrus reticulata, Citrus Sinensis, Pelargonium |
| (0.2% by vol) | gravea, Litsea Cubebe, Aurantium Bergamia, |
| | geraniol, Limonene and Lavandula angustiforlia. |

Formulation 2

| | |
|---|---|
| Orchid essences | Fitzroya cupressoide, Epidendrum secundum, |
| (5% by vol): | Oncidium altissimum and Oncidium sphacelatum |
| Secondary flower | Nelumbo nucifera, Rosa x, pruhoniciana, |
| essences | Echinocereus scheeri, Aloe vera, Prunella vulgaris, |
| (7% by vol.): | Jepunawa, Paradise lilastrum |
| Essential oils | Rosa centifloria absolute, Jasminum officinale abs, |
| (0.2% by vol) | Cananga adorata, Pelargonium gravea and Citrus |
| | Paradisi. |

Formulation 3

| | |
|---|---|
| Orchid essences | Fitzroya cupressoide, Angulo aclowesii and |
| (5% by vol): | Oncidium abortivum |
| Secondary flower | Rosa Moyesii, Strelitzia reginae, Passiflora |
| essences (7% by | bryonioides, Pilosocereus pachycladus, Prunella |
| vol.): | vulgaris, Aloe vera and Arnica montana |
| Essential oils | Rosa centifloria abs, Cananga odorata, Daucus |
| (0.2% by vol) | Carota, Lavendula Angustitolia, Pelargonium |
| | gravea and Citrus paradisi. |

Formulation 4 is a body oil formulation also made using conventional methods. The selected flower essences and essential oils in the amounts specified below were added drop wise to a base oil comprising Tahitian coconut oil with Gardenia flowers. Component parts are indicated in % amounts by volume, with the remainder of any solutions being made up with water.

Formulation 4

| | |
|---|---|
| Orchid essences | Fitzroya cupressoide, Angulo aclowesii, Oncidium |
| (10% by vol): | abortivum, Cattleya warscewiczii and Amazonas |
| Secondary flower | Rosa Moyesii, Strelitzia reginae, Passiflora |
| essences (5% by | bryonioides, Pilosocereus pachycladus, Prunella |
| vol.): | vulgaris, Aloe vera, Arnica montana, Rosa |
| | roxbourghii, Rosa moyesii, Orbeavariegata, |
| | Opzmtia cardiosperma and Lilium bulbiferum |
| Essential oils | Rosa centifloria abs, Cananga odorata, |
| (0.2% by vol) | Pelargonium gravea, Citrus paradisi and |
| | Cocosnucifera. |

Formulations 5, 6 and 7 are a spritzer and toner for face and body formulation also made using conventional methods. The selected flower essences and essential oils in the amounts specified below were added drop wise to a base flower water comprising of hydrolats of essential oils. Component parts are indicated in % amounts by volume, with the remainder of any solutions being made up with water.

Formulation 5: Spritzer and Toner

| | |
|---|---|
| Flower waters | Hydrolats of Citrus Reticulata |
| (86% by vol): | |
| Orchid essences | Fitzroya cupressoide, Epidendrum chioneum, |
| (5% by vol): | Miltonia phalaenopsis and Epidendrum Chioneum |
| Secondary flower | Arnica montana, Prunella vulgaris, Matriciaria |
| essences (7% by | chamomilla, Zinnia, elegans, Rosa |
| vol.): | pinosissima, Hibiscus, Rosa and Jepunbali |
| Essential oils | Citrus reticulata, Citrus Sinensis, Pelargonium |
| (0.2% by vol) | gravea, Litsea Cubebe, Aurantium Bergamia, |
| | geraniol, Limonene and Lavandula angustiforlia. |

Formulation 6: Spritzer and Toner

| | |
|---|---|
| Flower waters | Hydrolats of Rosa centifloria absolute |
| (86% by vol): | |
| Orchid essences | Fitzroya cupressoide, Epidendrum secundum, |
| (5% by vol): | Oncidium altissimum and Oncidiums phacelatum |
| Secondary flower | Nelumbo nucifera, Rosa x, pruhoniciana, |
| essences (7% by | Echinocereusscheeri, Aloe vera, Prunella vulgaris, |
| vol.): | Jepujawa, Paradise lilastrum |
| Essential oils | Rosa centifloria absolute, Jasminum officinale abs, |
| (0.2% by vol) | Cananga adorata, Pelargonium gravea and Citrus |
| | Paradisi. |

Formulation 7

| | |
|---|---|
| Flower waters | Hydrolats of Lavandula angustiforlia |
| (86% by vol): | |
| Orchid essences | Fitzroya cupressoide, Angulo aclowesii and |
| (5% by vol): | Oncidium abortivum |
| Secondary flower | Rosa Moyesii, Strelitzia reginae, Passiflora |
| essences (7% by | bryonioides, Pilosocereus pachycladus, Prunella |
| vol.): | vulgaris, Aloe vera and Arnica montana |
| Essential oils | Rosa centifloria abs, Cananga odorata, Daucus |
| (0.2% by vol) | Carota, Lavendula Angustitolia, Pelargonium |
| | gravea and Citrus paradisi. |

Formulation 8: Cleansing/Detox Gel

In a base of glycerin (vegetable), cera alba (beeswax) or candelilla wax, Prunus amygdalus dulcis (sweet almond) oil, coco-glucoside, oils of Rosa damascena (rose) oil, Citrus sinensis (orange) oil, Citrus reticulata (mandarin) oil, Pelgonium graveolens (geranium) oil, Litsea cubeba (Litsea) oil, Cinnammomum camphora (ho leaf) oil, geraniol (rose oil), flower essence of Epidendrum chioneum (sun orchid) Citrus limonum (lemon blossom).

Determination of the Efficacy of the Medicaments to Reduce Stress

The stress-relief efficacy of various medicaments was determined as follows. Test patients were chosen who were suffering from stress caused, for example, through illness, or as a result of their lifestyle or due to a temporary period of stress. Each one of Formulations 1 to 3 was formulated as an aqueous topical cream and was administered to the patient's face. Formulation 4 was administered to the whole body of the patient. Commercially available flower essence products, namely, “Bach Rescue Cream”, available for example, from Boots Plc and “FES Self Heal Cream”, available for example, from Tesco Plc or Wholefoods, were used as comparative formulations and were administered to the patient in accordance with the packaging instructions. The patients were then monitored over a one-month period and at the end of this period they were asked to give their opinions and observations on the performance of the formulation(s) administered to them. Comparative data for two known products is provided below.

| | Flower Therapy | |
|---|---|---|
| | composition of the | |
| | inventor | Boots ® |
| | Soft Orchid Body | Botanics |
| Product Attributes | Lotion | Hydrating Lotion |
| Appearance | 7.08 | 4.46 |
| Odour | 6.62 | 3.54 |
| Ease of Application | 7.23 | 5.15 |
| Ease of Absorbency | 7.00 | 4.77 |
| Absorbency time | 6.00 | 4.15 |
| Greasiness | 6.24 | 5.00 |
| (lower score if greasier) | | |
| Irritation | 5.00 | 5.00 |
| Skin Feel (softness) | 7.46 | 5.38 |
| Skin Moisturisation | 6.73 | 4.67 |

| | Flower Therapy | |
|---|---|---|
| | composition of the | Aveda® commercial |
| | inventor | equivalent |
| | Soft Orchid Body | Botanical |
| Product Attributes | Lotion | Hydrating Lotion |
| Appearance | 7.08 | 6.77 |
| Odour | 6.62 | 6.38 |
| Ease of Application | 7.23 | 6.69 |
| Ease of Absorbency | 7.00 | 6.38 |
| Absorbency time | 6.00 | 6.00 |
| Greasiness | 6.24 | 6.38 |
| (lower score if greasier) | | |
| Irritation | 5.00 | 5.00 |
| Skin Feel (softness) | 7.46 | 6.69 |
| Skin Moisturisation | 6.73 | 6.10 |

Results

The compositions provide improved cosmetics properties, particularly as a body lotion. Various compositions may be preferably formulated for use as a body lotion. Each patient/user expressed the view that the formulations helped them to become less stressed in comparison to using no cream/body oil and the comparative commercially available products commercially available non-orchid essence containing flower products. The improved cosmetics properties in better fulfilling user needs is considered a significant factor.

### CONCLUSIONS

As explained, various medicament formulations that can include one or more orchid essences can provide enhanced stress relief and mood enhancement compared with other commercially available non-orchid essence containing flower products. As explained, one or more plant extracts can be obtained and utilised. An extract may be a plant stem cell extract and may be preferably so in various embodiments. In various instances, an extract is a plant stem cell extract. As an example, a composition and/or a method may utilize one or more plant stem cell extracts.

1. A composition suitable for reducing or alleviating stress, the composition comprising one or more plant stem cell extracts in combination with one or more flower essences.

2. The composition according to claim 1, wherein the one or more plant stem cell extracts are selected from monocot and dicot flowering plants, shrubs and trees.

3. The composition according to claim 1, wherein the one or more flower essences are selected from Acineta superba, Amazonas, Angulo aclowesii, Caladenia latifolia, Cattleya warscewiczii, Cauleyatrianae, Chytroglossa aurata, Cymbidium lowianum, Epidendrum chioneum, Epidendrum prismatocarpum, Epidendrum secundum, Fitzroya cupressoide, Hofmeister ellaru microscopica, Laeliocattleya anceps clara, Laeliocattleya hybr., Miltonia phalaenopsis, Oncidium abortivum, Oncidium altissimum, Oncidium incurvum, Oncidium lanceanum, Oncidium sphacelatum, Orchis mascula, Paphiopedilum harrysianum, Paphiopedilum insigne, Stanhopeawardii, Vanda tricolor, Victoria amazonica and combinations thereof.

4. The composition according to claim 3, wherein the one or more flower essences are selected from Amazonas, Angulo aclowesii, Caladenia latifolia, Cattleya warscewiczii, Chytroglossa aurata, Epidendrum chioneum, Epidendrum secundum, Fitzroya cupressoide, Hofmeister ellaru microscopica, Miltonia phalaenopsis, Oncidium abortivum, Oncidium sphacelatum and combinations thereof.

5. The composition according to claim 1, further comprising one or more secondary flower essences selected from Achillea millefolium, Aesculus hippocastanum, Allium angulosum, Allium ursinum, Aloe striata, Aloe vera, Anagallis arvensis, Anethum graveolens, Arnica montana, Artemisia arvensis, Bellis perennis, Borago officinalis, Bromus ramosus, Canarina canariensis, Capsicum annuum, Caralluma russelliana, Celendula officinalis, Cereus peruvianus, Ceropegiafusca, Chinensis viridijlora, Cistus albidus, Cistus allidus, Citrus limon, Cleistocactus ritterei, Cleistocactus stausii, Cocos nucifera, Cucurbita pepo, Daucus carota, Dicentra spectabilis, Echinaecea purpurea, Echinocactus grusonii, Echinocereus scheeri, Echinopsis oxygona, Echium vulgare, Echium wildpretii, Epilobium angustifolium, Erica arborea, Eucalyptus globules, Euphorbia pulcherrima, Ferocactus schwarzii, Geranium perforatum, Ginkgo biloba, Helianthus annuus, Hibiscus rosa sinensis, Hibiscus, Hylocereus undatus, Hypericum perforatum, Ipomea purpurea, Iris germanica, Jepujawa, Jepunbali, Jepunjawa, Jepunawa, K9, Lavandula officinalis, Lilium bulbiferum, Mammillaria rubrograndis, Matricaria chamomilla, Mentha pulegium, Mimosa pudica, Monstera deliciosa, Musa x paradisiacal, Myitillocactus geometrizans, Myosotis sylvatica, Narcissus tazetta, Nelumbo nucifera, Ocimum basilicum, Oenothera biennis, Opuntia cardiosperma, Opuntia dejecta, Opzmtia cardiosperma, Orbeavariegata, Paradisea liliastrum, Passiflora bryonioides, Pilosocereus pachycladus, Plumbago auriculata, Polygonum bistorta, Primula veris, pruhoniciana, Prunella vulgaris, Prunus amygdalus, Ranunculus acris, Rosa canina, Rosa majalis, Rosa moyesii, Rosa pinosissima, Rosa roxbourghii, Rosa rugosa “Sara van fleet”, Rosa rugosa “Souvenir de philemoncochet”, Rosa spinosissima, Rosa x pruhoniciana, Rosa, Rosmarinus officinalis, RQ5 and RQ8, Rubus fructicosus, Salix vitellina, Salvia officinalis, Selenicereus grandiflorus, Seticereus icosagonus, Sonchus acaulis, Stapelia deshietiana, Stenocereus marginatus, Stopelia desmetiana, Strelitzia reginae, Taraxacu officinale, Trifolium pratense, Tussilago farara, Valeriano officinalis, Verbascum thapsus, Vinca minor, Viola hirta, Viscum album, Zantedeschia aethiopica, Zea mays, Zinnia elegans and combinations thereof.

6. The composition according to claim 5, wherein the one or more secondary flower essences are selected from Aloe vera, Arnica montana, Echinocereus scheeri, Hibiscus, Jepujawa, Jepunbali, Jepunawa, Lilium bulbiferum, Matriciaria chamomilla, Nelumbo nucifera, Opzmtia cardiosperma, Orbea variegata, Paradise lilastrum, Passiflora bryonioides, Pilosocereus pachycladus, pruhoniciana, Prunella vulgaris, Rosa, Rosa moyesii, Rosa pinosissima, Rosa roxbourghii, Rosa xpruhoniciana, Strelitzia reginae, Zinnia elegans and combinations thereof.

7. The composition according to claim 1, further comprising one or more essential oils selected from Aurantium Bergamia, Buswellia sp., Cananga adorata, Cananga odorata, Chamaemelum, Citrus aurantifolia, Citrus aurantium, Citrus paradisi, Citrus reticulata, Citrus sinesnsis, Cocos nucifera, Cymbopogan martini, Cymbopogon citrates, Daucus carota, geraniol, Jasminum officinale abs, Lavandula angustiforlia, Limonene, Litseacubebe, Melissa officinalis, Pelargonium gravea, Ravensara aromatica, riganum, Rosa centifloria absolute, Santalum album, Santalum spicatum and combinations thereof.

8. The composition according to claim 1, wherein the amount of the one or more flower essences is present in the range 0.5-15% by volume.

9. The composition according to of claim 1, wherein the amount of the one or more flower essences is present in the range 2-10% by volume.

10. The composition according to claim 5, wherein the amount of the one or more secondary flower essences is present in the range 0.5-15% by volume.

11. The composition according to claim 5, wherein the amount of the one or more secondary flower essences is present in the range 2-10% by volume.

12. The composition according to claim 7, wherein the amount of the one or more essential oils is present in the range 0.05-5% by volume.

13. The composition according to claim 7, wherein the amount of the one or more essential oils is present in the range 0.05-3% by volume.

14. The composition according to claim 7, wherein the amount of the one or more essential oils is present in the range 0.1-1% by volume.

15. The composition according to claim 1, wherein the composition is an aqueous-based extract.

16. The composition according to claim 1, wherein the composition is a cream.

17. The composition according to claim 1, wherein the composition includes one or more optional berry extracts including, but not limited to, extracts of Maiqui berries.

18. The composition according to claim 1, wherein the composition comprises Fitzroya cupressoide.

19. The composition according to claim 1, wherein the composition consists of Fitzroya cupressoide in a defined plant extract.

20. The composition according to claim 18, wherein the composition does not comprise stem cells, such as those to derive the one or more plant stem cell extracts.
